(19) United States
(12) Patent Application Publication
(10) Pub. No.: US 2002/0182615 A1
(43) Pub. Date: Dec. 5, 2002

(54) All in one nucleic acid amplification assay

(76) Inventors: Michael B. Cerney, Libertyville, IL (US); Jon D. Kratochvil, Kenosha, WI (US); Thomas G. Laffler, Libertyville, IL (US); Ronald L. Marshall, Zion, IL (US); Joann C. Sustachek, Racine, WI (US)

Correspondence Address: Steven F. Weinstock, Abbott Laboratories, Dept. 377 - AP6D-2, 100 Abbott Park Road, Abbott Park, IL, 60064-6050, US

(21) Appl. No.: 10/001,211

(22) Filed: Oct. 24, 2001

Related U.S. Application Data
(62) Division of application No. 08/514,704, filed on Aug. 14, 1995, now abandoned.

(51) Int. Cl. C12Q 1/68; C12P 19/34

(52) U.S. Cl. 435/6; 435/91.2

(57) ABSTRACT

A method for detecting a target nucleic acid sequence sequence using nucleic acid amplification wherein hybridization probes for detection of the amplified target sequence are present during the amplification reaction.

### RELATED APPLICATION

This application is filed contemporaneously with commonly owned U.S. Pat. application Ser. No.. 08/514,704 (Docket No. 5791.US.01) the full text of which is hereby incorporated by reference.

### FIELD OF THE INVENTION

The present invention relates to nucleic acid amplification assays and, in particular, relates to nucleic acid amplification assays in which amplification via primer extension is carried out in the presence of hybridization probes which have different sequence than the extension primers, and which are complementary to all or a portion of an intended target sequence.

### BACKGROUND OF THE INVENTION

Methods for amplifying and detecting a target nucleic acid sequence that may be present in a test sample are, by now, well known in the art. Such methods include the polymerase chain reaction (PCR) which has been described in U.S. Pat. Nos. 4,683,195 and 4,683,202, the ligase chain reaction (LCR) described in EP-A-320 308, gap LCR (GLCR) described in European Patent Application EP-A-439 182, multiplex LCR described in International Patent Application No. WO 93/20227 and the like. These methods have found widespread application in the medical diagnostic field as well as the fields of genetics, molecular biology and biochemistry.

While amplification reactions generally are sensitive, one drawback particularly associated with PCR is that it can be non-specific. In other words, PCR is known to amplify target sequences as well as non-target sequences. However, this drawback can be remedied by distinguishing the amplified target sequences from amplified non-target sequences using an internal hybridization probe. Internal hybridization probes generally are nucleic acid sequences which are complementary to a region of the target sequence which is different from the regions which are complementary to the primer sequences. Such sequences can be employed to hybridize with the amplification products and distinguish target and non-target amplification products. Given that the internal hybridization probes are selected to hybridize with a non-primer region of the amplification products, they confer an additional level of specificity upon PCR.

For example, a solid phase coated with an internal hybridization probe can be contacted with PCR reaction products (variably referred to as an amplicon) which may contain both target and non-target sequences. However, under appropriate hybridization conditions the internal hybridization probe will bind to amplified target but not amplified non-target sequences. Hence, the target sequences and non-target sequences can be separated from each other and the target sequences can then be detected. This type of capture and detection is generally effective at discriminating amplified target sequences from amplified non-target sequences.

Unfortunately, however, this type of assay has generally been accomplished by forming the probe/amplicon hybrids in separate areas or vessels. It is therefore necessary to transfer reagents and reactants between these areas which gives rise to the possibility of contamination. Given the ability of an amplification reaction to generate copies of a target sequence, contamination poses a serious threat to the reliability of these reactions. For example, even if a single extraneous target sequence contaminates a test sample that is otherwise devoid of target sequence, the single extraneous target sequence can give rise to a false positive result. Contamination poses a particular threat in clinical settings where multiple test samples are assayed in close proximity.

There thus is a need for a specific amplification reaction which minimizes the threat of contamination and is readily amenable to automation.

### SUMMARY OF THE INVENTION

The present invention is a method for detecting a target nucleic acid sequence comprising the steps of:

(a) forming a reaction mixture comprising nucleic acid amplification reagents, a hybridization probe, and a test sample suspected of containing nucleic acid comprising said target nucleic acid sequence, wherein

(i) said amplification reagents comprise an amplification primer comprising a nucleic acid sequence complementary to and capable of hybridizing with said sample nucleic acid such that the primer, upon extension by said amplification reagents, results in a nucleic acid comprising a nucleic acid sequence complementary to said target sequence, or complementary to a portion thereof;

(ii) said hybridization probe comprising a non-extendible nucleic acid sequence wherein said nucleic acid sequence (a) is complementary to a portion of said sequence complementary to said target sequence and (b) is different from said primer nucleic acid sequence;

(b) subjecting said mixture to amplification conditions to generate at least one said nucleic acid comprising a nucleic acid sequence complementary to said target sequence;

(c) hybridizing said probe to said nucleic acid comprising a nucleic acid sequence complementary to said target sequence, so as to form a complex comprising said probe and said nucleic acid; and

(d) detecting the presence of said complex as an indication of the presence of said target sequence in said sample.

The invention is also directed to a kit for detecting the presence of a nucleic acid sequence in a test sample comprising one or more suitable containers containing:

(a) a non-extendible hybridization probe which is complementary to said target sequence's complementary sequence; and

(b) an amplification primer which is complementary to said target sequence.

The method disclosed herein provides a method of detecting a target nucleic acid sequence in a test sample which allows for specific detection of a target nucleic acid sequence. Advantageously, the production of a double stranded product which can be detected may be achieved in a single reaction vessel. As a result, the threat of contamination is minimized and the method is readily amenable to automation.

The method of the present invention generally is suitable for use with nucleic acid amplification reactions and is particularly well suited for use with PCR.

Preferably, the amplification primer nucleic acid sequence which is complementary to a region of the target sequence carries either a detection label or a capture label. Alternatively, the primer may not carry a label at all, but the primers extension product, through the process of adding detectable deoxynucleotide triphosphates, can be functionalized with a detection label or a capture label. Similarly, the hybridization probe preferably carries either a detection label or a capture label. The primer and probe are distinguishable in that the probe is non-extendible and has a melt temperature that is distinct from the primer sequence.

The hybridization probe and the amplification primer or its extension product typically carry distinct types of labels. Thus, when the amplification primer or its extension product carries a detection label, the hybridization probe will carry a capture label, and when the amplification primer or its extension product carries a capture label, the hybridization probe will carry a detection label. Standard heterogeneous immunoassay techniques can be employed to detect the probe/single stranded amplicon member complexes.

The method of the present invention is also suitable for use with other amplification techniques such as NASBA and strand displacement amplification. These and other amplification methods are generally discussed in Wolcott Advances in Nucleic Acid based Detection Methods, Clin. Microbiology Reviews, Vol 5, No. 4 pp370-386 (1992) incorporated herein by reference.

### BRIEF DESCRIPTION OF THE DRAWINGS

FIGS. 1(a)-(e) are a schematic representation of an embodiment of the method provided herein.

### DETAILED DESCRIPTION OF THE INVENTION

The present invention generally comprises the steps of contacting a test sample suspected of containing a target nucleic acid sequence with amplification reaction reagents comprising an amplification primer, and a hybridization probe that can hybridize with an internal region of the amplicon sequences. Probes and primers employed according to the method herein provided are labeled with capture and detection labels wherein probes are labeled with one type of label and primers are labeled with the other type of label. Additionally, the primers and probes are selected such that the probe sequence has a lower melt temperature than the primer sequences. The amplification reagents, hybridization probe and test sample are placed under amplification conditions whereby, in the presence of target sequence, copies of the target sequence (an amplicon) are produced at temperatures above the Tm of the probe(s). In the usual case, the amplicon is double stranded because primers are provided to amplify a target sequence and its complementary strand. The double stranded amplicon is then thermally denatured to produce single stranded amplicon members.

After denaturation, the mixture is cooled, i.e., re-natured, to enable the formation of complexes between the probes and single stranded amplicon members. The rate of temperature reduction from the denaturation temperature down to a temperature at which the probes will bind to single stranded amplicons is preferably quite rapid (for example 8 to 15 minutes) and particularly through the temperature range in which the polymerase enzyme is active for primer extension. We have discovered that this rapid cooling not only prevents extension of any primer which may have reattached to single stranded amplicons during cooling, but also results in a substantial amount of probe binding to single stranded amplicon. We found it particularly surprising that, after rapid cooling to a temperature which was below the Tm of both the probes and the single stranded amplicons, we were able to readily detect the complexes formed by hybridization probes and single stranded amplicons. Because the melt temperature of the single stranded amplicon produced by the primers is higher than the melt temperature of the probes, we would have expected that, as the mixture was cooled, the re-formation of the double stranded amplicon would be the more likely binding event and that as the temperature is lowered to a Tm which below both the single stranded amplicon and the probe, the amplicons would compete with the probea, and either prevent them from binding with the single stranded amplicon altogether, or displace the probes once they had bound. Instead we have discovered that upon rapid cooling of the amplification mixture, the probes are able to bind the single stranded amplicon members to a degree which is sufficient for detection of the probe/amplicon complexes. Apparently, as the probes and single stranded amplicons are cooled, the probes actually bind preferentially. This preferential binding is particularly surprising insofar as it occurs even when the primer sequences are present in excess of the probes.

The present invention makes possible nucleic acid amplification assays in which hybridization probes required for detection are already present throughout the amplification reaction, thereby eliminating the need to open the reaction vessel for the purpose of adding a detection probe.

After the probe/single stranded amplicon member hybrids are formed, they are detected. Standard heterogeneous assay formats are suitable for detecting the hybrids using the detection labels and capture labels present on the primers and probes. The hybrids can be bound to a solid phase reagent by virtue of the capture label and detected by virtue of the detection label. In cases where the detection label is directly detectable, the presence of the hybrids on the solid phase can be detected by causing the label to produce a detectable signal, if necessary, and detecting the signal. In cases where the label is not directly detectable, the captured hybrids can be contacted with a conjugate, which generally comprises a binding member attached to a directly detectable label. The conjugate becomes bound to the complexes and the conjugates presence on the complexes can be detected with the directly detectable label. Thus, the presence of the hybrids on the solid phase reagent can be determined. Those skilled in the art will recognize that wash steps may be employed to wash away unhybridized amplicon or probe as well as unbound conjugate.

A test sample is typically anything suspected of containing a target sequence. Test samples can be prepared using methodologies well known in the art such as by taking a specimen from a patient and, if necessary, disrupting any cells contained therein to release nucleic acids. For ease of explanation, throughout this disclosure the target sequence is described as single stranded. However, this is intended to include the case where the target sequence is actually double stranded but is merely separated from its complement prior to hybridization with the amplification primer sequences. In the case where PCR is employed in the instant method, the ends of the target sequences are usually known, and in cases where LCR or a modification thereof is employed in the instant method, the entire target sequence is usually known. Typically, the target sequence is a nucleic acid sequence such as for example RNA or DNA.

The method provided herein finds utility in well known amplification reactions which thermal cycle reaction mixtures particularly PCR and GLCR. Amplification reactions typically employ primers to repeatedly generate copies of a target nucleic acid sequence which is usually a small region of a much larger nucleic acid sequence. Primers are themselves nucleic acid sequences that are complementary to regions of a target sequence and under amplification conditions, hybridize or bind to the complementary regions of the target sequence. Copies of the target sequence are typically generated by the process of primer extension and/or ligation which utilizes enzymes with polymerase or ligase activity, separately or in combination, to add nucleotides to the hybridized primers and/or ligate adjacent primer pairs. The nucleotides that are added to the primers as monomers or preformed oligomers, are also complementary to the target sequence. Once the primers have been sufficiently extended and/or ligated they are separated from the target sequence, for example, by heating the reaction mixture to a “melt temperature” which is one where complementary nucleic acid strands dissociate. Thus, a sequence complementary to the target sequence is formed.

A new amplification cycle can then take place to further amplify the number of target sequences by separating any double stranded sequences, allowing primers to hybridize to their respective targets, extending and/or ligating the hybridized primers and re-separating. The complementary sequences that are generated by amplification cycles can serve as templates for primer or probe extension to further amplify the number of target sequences. Typically, a reaction mixture is cycled between 15 and 100 times, more typically, a reaction mixture is cycled between 25 and 50 times. In this manner, multiple copies of the target sequence and its complementary sequence are produced. Thus, under amplification conditions primers initiate amplification of the target sequence when it is present.

Generally, two primers which are complementary to a portion of a target strand and its complement are employed in PCR. For LCR, four primers, two of which are complementary to a target sequence and two of which are similarly complementary to the targets complement, are generally employed. In addition to the primer sets and enzymes previously mentioned, a nucleic acid amplification reaction mixture may also comprise other reagents which are well known and include but are not limited to: enzyme cofactors such as manganese; magnesium; salts; nicotinamide adenine dinucleotide (NAD); and deoxynucleotide triphosphates (dNTPs) such as for example deoxyadenine triphosphate, deoxyguanine triphosphate, deoxycytosine triphosphate and deoxythymine triphosphate.

While the amplification primers initiate amplification of the target sequence, the hybridization probe is not involved in amplification. Hybridization probes are generally nucleic acid sequences or uncharged nucleic acid analogs such as, for example peptide nucleic acids which are disclosed in International Patent Application WO 92/20702; morpholino analogs which are described in U.S. Pat. Nos. 5,185,444, 5,034,506, and 5,142,047; and the like. Depending upon the type of label carried by the probe, the probe is employed to capture or detect the amplicon generated by the amplification reaction. The probe is not involved in amplification of the target sequence and therefor may have to be rendered “non-extendible” which means that additional dNTPs cannot be added to the probe. In and of themselves analogs usually are non-extendible, nucleic acid probes, however, can be rendered non-extendible. Nucleic acid probes can be rendered non-extendible by modifying the 3′ end of the probe such that the hydroxyl group is no longer capable of participating in elongation. For example, the 3′ end of the probe can be functionalized with the capture or detection label to thereby consume or otherwise block the hydroxyl group. Alternatively, the 3′ hydroxyl group simply can be cleaved, replaced or modified. U.S. Pat. application Ser. No. 07/049,061 filed Apr. 19, 1993 describes modifications which can be used render a probe non-extendible.

According to the method provided herein, the ratio of primers to probes is not important. Thus, either the probes or primers can be added to the reaction mixture in excess whereby the concentration of one would be greater than the concentration of the other. Alternatively, primers and probes can be employed at in equivalent concentrations. Preferably, however, the primers are added to the reaction mixture in excess of the probes. Thus, primer to probe ratios of, for example, 5:1 and 20:1 are preferred according to the instant invention.

While the length of the primers and probes can vary, as mentioned before, the probe sequences are selected such that they have a lower melt temperature than the primer sequences. Hence, the primer sequences are generally longer than the probe sequences. Typically, the primer sequences are in the range of between 20 and 50 nucleotides long, more typically in the range of between 25 and 30 nucleotides long. The typical probe is in the range of between 10 and 25 nucleotides long, more typically between 15 and 20 nucleotides long.

Various methods for synthesizing primers and probes are well known in the art. Similarly, methods for attaching labels to primers or probes are also well known in the art. For example, it is a matter of routine to synthesize desired nucleic acid primers or probes using conventional nucleotide phosphoramidite chemistry and instruments available from Applied Biosystems, Inc., (Foster City, Calif.), Dupont (Wilmington, Del.), or Perseptive (Bedford, Mass.). Many methods have been described for labeling oligonucleotides such as the primers or probes of the present invention. Enzo Biochemical (New York) and Clontech (Palo Alto) both have described and commercialized probe labeling techniques. For example, a primary amine can be attached to a 3′ oligo terminus using 3′-Amine-ON CPG™ (Clontech, Palo Alto, Calif.). Similarly, a primary amine can be attached to a 5′ oligo terminus using Aminomodifier II® (Clontech). The amines can be reacted to various haptens using conventional activation and linking chemistries. In addition, copending applications U.S. Ser. Nos. 625,566, filed Dec. 11, 1990 and 630,908, filed Dec. 20, 1990 teach methods for labeling probes at their 5′ and 3′ termini, respectively.

Publications WO 92/10505, published Jun. 25, 1992 and WO 92/11388 published Jul. 9, 1992 teach methods for labeling probes at their 5′ and 3′ ends respectively. According to one known method for labeling an oligonucleotide, a label-phosphoramidite reagent is prepared and used to add the label to the oligonucleotide during its synthesis. For example, see Thuong, N. T. et al., Tet. Letters, 29(46):5905-5908 (1988); or Cohen, J. S. et al., published U.S. Pat. application Ser. No. 07/246,688 (NTIS ORDER No. PAT-APPL-7-246,688) (1989). Preferably, probes are labeled at their 3′ and 5′ ends.

Solid phase reagents typically comprise “specific binding members” bound to a solid phase. As used herein, specific binding member means a member of a binding pair, i.e., two different molecules where one of the molecules through, for example, chemical or physical means specifically binds to the other molecule. In addition to antigen and antibody specific binding pairs, other specific binding pairs include, but are not intended to be limited to, avidin and biotin; haptens such as adamantane and carbazole which are described in U.S. patent application Ser. No. 08/049,888 filed Apr. 21, 1993, and U.S. patent application Ser. No. 08/084,495 filed Jul. 1, 1993, respectively and antibodies specific for haptens; complementary nucleotide sequences; complementary nucleic acid analogs such as those previously mentioned; enzyme cofactors or substrates and enzymes; and the like.

Solid phase refers to any material which is insoluble, or can be made insoluble by a subsequent reaction. The solid phase can be chosen for its intrinsic ability to attract and immobilize a binding member to form a solid phase reagent. Alternatively, the solid phase can retain an additional receptor which has the ability to attract and immobilize a binding member to form a solid phase reagent. The additional receptor can include a charged substance that is oppositely charged with respect to a binding member or to a charged substance conjugated to a binding member. As yet another alternative, the receptor molecule can be any specific binding member which is immobilized upon (attached to) the solid phase and which has the ability to immobilize another binding member through a specific binding reaction. The receptor molecule enables the indirect binding of a binding member to a solid phase material before the performance of the assay or during the performance of the assay. The solid phase thus can be, for example, latex, plastic, derivatized plastic, magnetic or non-magnetic metal, glass or silicon surface or surfaces of test tubes, microtiter wells, sheets, beads, microparticles, chips, and other configurations known to those of ordinary skill in the art. It is contemplated and within the scope of the invention that the solid phase also can comprise any suitable porous material with sufficient porosity to allow, when necessary, access by a conjugate. Microporous structures are generally preferred, but materials with gel structure in the hydrated state may be used as well. The porous structure of nitrocellulose has excellent absorption and adsorption qualities for a wide variety of reagents including binding members. Nylon also possesses similar characteristics and also is suitable. Such materials may be used in suitable shapes, such as films, sheets, or plates, or they may be coated onto or bonded or laminated to appropriate inert carriers, such as paper, glass, plastic films, or fabrics. The method by which a binding member is attached to a solid phase can be selected from any of the conventional methods and is a matter of choice for one skilled in the art.

Capture labels are carried by the primers or probes and can be a specific binding member which forms a binding pair with the solid phase reagent's specific binding member. It will be understood, of course that the primer or probe itself may serve as the capture label. For example, in the case where a solid phase reagent's binding member is a nucleic acid sequence, it may be selected such that it binds a complementary portion of the primer or probe to thereby immobilize the primer or probe to the solid phase. In cases where the probe itself serves as the binding member, those skilled in the art will recognize that the probe will contain a sequence or “tail” that is not complementary to the single stranded amplicon members. In the case where the primer itself serves as the capture label, at least a portion of the primer will be free to hybridize with a nucleic acid on a solid phase because the probe is selected such that it is not fully complementary to the primer sequence.

The term detection label refers to a molecule or moiety having a property or characteristic which is capable of detection. A detection label can be directly detectable, as with, for example, radioisotopes, fluorophores, chemiluminophores, enzymes, colloidal particles, fluorescent microparticles and the like; or a label may be indirectly detectable, as with, for example, specific binding members. It will be understood that direct labels may require additional components such as, for example, substrates, triggering reagents, light, and the like to enable detection of the label. As previously mentioned, when indirect labels are used for detection, they are typically used in combination with a conjugate. A conjugate is typically a specific binding member which has been attached or coupled to a directly detectable label. Similarly to the synthesis of solid phase reagents, coupling chemistries for synthesizing a conjugate are well known in the art and can include, for example, any chemical means and/or physical means that does not destroy the specific binding property of the specific binding member or the detectable property of the label.

Generally, probe/single stranded amplicon member complexes can be detected using techniques commonly employed to perform heterogeneous immunoassays. Preferably, detection is performed according to the protocols used by the commercially available Abbott LCx® instrumentation (Abbott Laboratories; Abbott Park, Ill.)

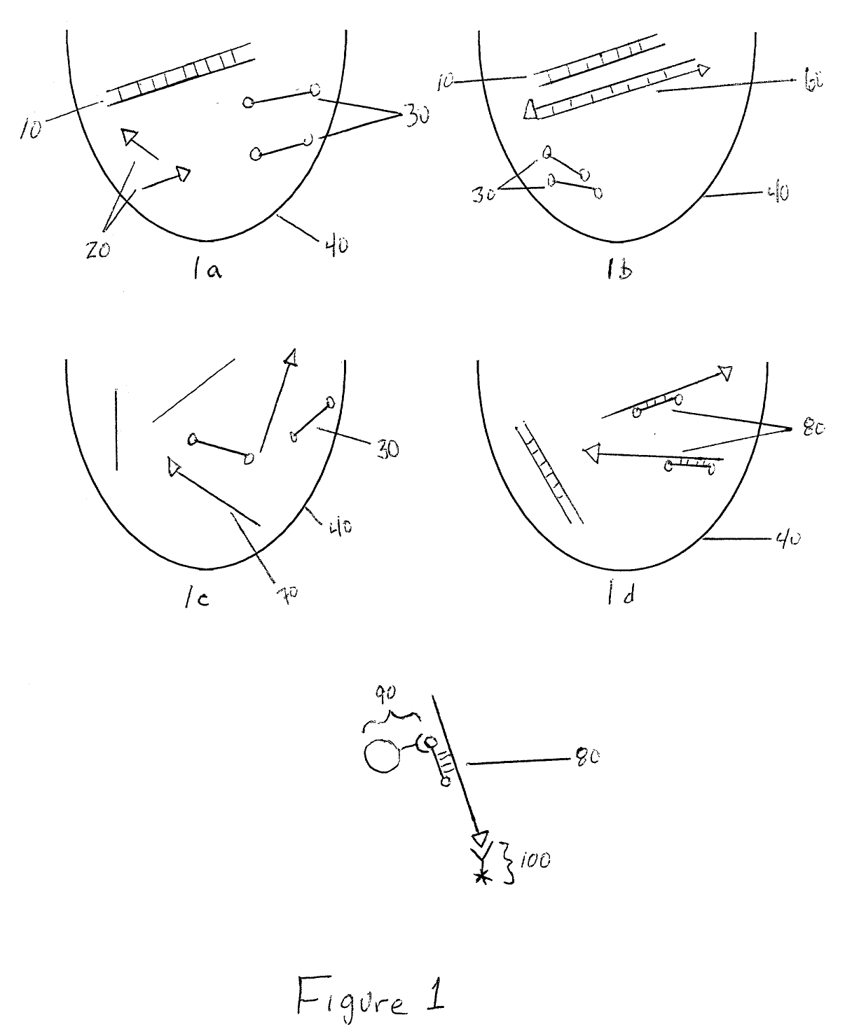

An embodiment of the invention will now be described in accordance with FIG. 1(a)-FIG. 1(e). As shown by FIG. 1(a), a test sample containing target sequence 10, amplification reagents comprising primers 20, and hybridization probes 30 are added to vessel 40 to form a reaction mixture. After the addition of the reagents, the reaction mixture is subjected to amplification conditions so that a copy of the target strand 60 is produced as shown in FIG. 1(b). The mixture of FIG. 1(b) can then be heated to thermally dissociate the double stranded amplicon as shown in FIG. 1(c) which illustrates single stranded amplicon members 70. The mixture of FIG. 1(c) is then cooled and probes 30 bind the single stranded amplicon members 70 to form probe/single stranded amplicon member complexes 80 shown in FIG. 1(d). FIG. 1(e) illustrates a method of detecting the complexes. As shown by FIG. 1(e), the complexes are immobilized to solid phase reagent 90, and conjugate 100 is immobilized to the complexes. The presence of the complexes on the solid phase reagent can then be detected as an indication of the presence of the target sequence in the test sample.

The following examples are provided to further illustrate the present invention and not intended to limit the invention.

### Examples

While the invention has been described in detail and with reference to specific embodiments, it will be apparent to one skilled in the art that various changes and modifications may be made to such embodiments without departing from the spirit and scope of the invention. Additionally, all patents and publications mentioned above are herein incorporated by reference.

The following examples demonstrate use of the present invention for the detection hepatitis GB virus using novel hepatitis GB virus (HGBV) DNA oligomer primers and probes. These DNA primers and probes are identified as SEQUENCE ID NO 1,SEQUENCE ID NO 2, SEQUENCE ID NO 3, SEQUENCE ID NO 4, SEQUENCE ID NO 5, SEQUENCE ID NO 6, SEQUENCE ID NO 7, SEQUENCE ID NO 8, SEQUENCE ID NO 9, SEQUENCE ID NO 10, SEQUENCE ID NO 11, SEQUENCE ID NO 12, SEQUENCE ID NO 13, SEQUENCE ID NO 14 and SEQUENCE ID NO 15. SEQUENCE ID NO 1, 2, 3, 4 and 5 are specific for the 5′ non-translated region (NTR) of HGBV. SEQUENCE ID NO 6, 7, 8, 9, 10, 11, 12, 13, 14 and 15 are specific for the NS3 region of HGBV. The HGBV primers specific for the 5′ NTR region of HGBV are SEQUENCE ID NO 1 and SEQUENCE ID NO 2. The HGBV primers specific for the NS3 region of HGBV are SEQUENCE ID NO 6 and SEQUENCE ID NO 7.

### Example 1

### Amplification with HGBV NTR Primer Set

Target-specific primer detection probes were designed to detect the above target sequence by oligonucleotide hybridization PCR. These primers were SEQUENCE ID NO 1 and SEQUENCE ID NO 2.

A. NTR Primer Set. Target sequences were amplified using the NTR primer set (SEQUENCE ID NO 1 and SEQUENCE ID NO 2) and haptenated with adamantane at their 5′ end using standard cyanoethyl phosphoramidite coupling chemistry.

The amplified product then was detected using different hybridization probes as shown in TABLE 1. Reactivity was assessed using human placental DNA (hp DNA; Sigma, St. Louis, Mo.).

TABLE 1

| Reaction | Target | Primer | Detection Probe |
|---|---|---|---|
| 1 | 1 μg hpDNA | NTR S1/NTR A1 | NTR CJ1 |
| | | (SEQ ID NO 1/ | (SEQ ID NO 3) |
| | | SEQ ID NO 2) | |
| 2 | 2 ng NTR Plasmid | NTR S1/NTR A1 | NTR CJ1 |
| | | (SEQ ID NO 1/ | (SEQ ID NO 3) |
| | | SEQ ID NO 2) | |
| 3 | 4 ng NTR Plasmid | NTR S1/NTR A1 | NTR CJ1 |
| | | (SEQ ID NO 1/ | (SEQ ID NO 3) |
| | | SEQ ID NO 2) | |
| 4 | 1 μg hpDNA | NTR S1/NTR A1 | NTR CJ2 |
| | | (SEQ ID NO 1/ | (SEQ ID NO 4) |
| | | SEQ ID NO 2) | |
| 5 | 2 ng NTR Plasmid | NTR S1/NTR A1 | NTR CJ2 |
| | | (SEQ ID NO 1/ | (SEQ ID NO 3) |
| | | SEQ ID NO 2) | |
| 6 | 4 ng NTR Plasmid | NTR S1/NTR A1 | NTR CJ2 |
| | | (SEQ ID NO 1/ | (SEQ ID NO 4) |
| | | SEQ ID NO 2) | |
| 7 | 1 μg hpDNA | NTR S1/NTR A1 | NTR RM1 |
| | | (SEQ ID NO 1/ | (SEQ ID NO 5) |
| | | SEQ ID NO 2) | |
| 8 | 2 ng NTR Plasmid | NTR S1/NTR A1 | NTR RM1 |
| | | (SEQ ID NO 1/ | (SEQ ID NO 5) |
| | | SEQ ID NO 2) | |
| 9 | 4 ng NTR Plasmid | NTR S1/NTR A1 | NTR RM1 |
| | | (SEQ ID NO 1/ | |
| | | SEQ ID NO 2) | |
| 10 | 1 μg hpDNA | NTR S1/NTR A1 | NTR CJ1/CJ2/RM1 |
| | | (SEQ ID NO 1/ | (SEQ ID NO 3/ |
| | | SEQ ID NO 2) | SEQ ID NO 4/ |
| | | | SEQ ID NO 5) |
| 11 | 2 ng NTR Plasmid | NTR S1/NTR A1 | NTR CJ1/CJ2/RM1 |
| | | (SEQ ID NO 1/ | (SEQ ID NO 3/ |
| | | SEQ ID NO 2) | SEQ ID NO 4/ |
| | | | SEQ ID NO 5) |
| 12 | 4 ng NTR Plasmid | NTR S1/NTR A1 | NTR CJ1/CJ2/RM1 |
| | | (SEQ ID NO 1/ | (SEQ ID NO 3/ |
| | | SEQ ID NO 2) | SEQ ID NO 4/ |
| | | | SEQ ID NO 5) |
| * The NTR plasmid (Clone pHGBV-C clone #1) was deposited at the American Type Culture Collection, 12301 Parklawn Drive, Rockville, Maryland 20852 as of Nov. 8, 1994, under the terms of the Budapest Treaty and will be maintained for a period of thirty (30) years from the date of deposit, or for five (5) years after the last request for the deposit, or for the enforceable period of the U.S. Pat., whichever is longer. | | | |
| # The deposits and any other deposited material described herein are provided for convenience only and are not required to practice the present invention in view of the teachings provided herein. pHGBV-C clone #1 was accorded A.T.C.C. Deposit No. 69711. The HGBV cDNA sequences in all of the deposited materials are incorporated herein by reference. | | | |

B. Description of plasmid. PCR extension was performed for reactions 1-12 (see Table 1) as described below using the 10×PCR buffer (Perkin Elmer, Foster City, Calif.) which consisted of 100 mM Tris-HCl, pH 8.3, 500 mM KCl. The MgCl2 final concentration was 2 mM and the final concentration of the nucleotides was @ 200 μM. The reaction conditions for Table 1 are shown in Table 2.

TABLE 2

| | Primer | Probe | Enzyme |
|---|---|---|---|
| Reactions | Concentration | Concentration | Concentration |
| 1-12 | 0.25 μM | 0.01 μM | 10 U Taq |

* Reactions were amplified as follows: 95° C. 2′ 1 cycle; 94° C.
1′/55° C. 1′/72° C. 1′ 30 cycles; 95° C. 5′ , 15° C. soak. After
maintaining the reaction mixture at 95° C. for 5 minutes,
probe hybridization was accomplished according to the
present invention by lowering the temperature of the
amplification reaction 95° to 15° in 11 minutes
according to the following regimen:

| ° C. | Time |
|---|---|
| 97 | 0 |
| 90 | 11 |
| 80 | 16 |
| 75 | 21 |
| 70 | 25 |
| 65 | 29 |
| 60 | 34 |
| 55 | 40 |
| 50 | 48 |
| 45 | 57 |
| 40 | 1′11″ |
| 35 | 1′35″ |
| 30 | 2′34″ |
| 25 | 4′43″ |
| 20 | 7′33″ |
| 18 | 8′43″ |
| 17 | 9′22″ |
| 16 | 10′ |
| 15 | 10′56″ |

Following amplification, reaction products were detected on the Abbott LCx® system (available from Abbott Laboratories, Abbott Park, Ill.). The data from these experiments are presented in TABLE 3. The data in TABLE 3 demonstrated specific amplification and detection of the HGBV target sequence.

TABLE 3

| Reaction* | LCx ® (c/s/s) | Reactivity |
|---|---|---|
| 1 | 14.7 | Nonreactive |
| 2 | 725.5 | Reactive |
| 3 | 776.2 | Reactive |
| 4 | 12.4 | Nonreactive |
| 5 | 703.8 | Reactive |
| 6 | 772.4 | Reactive |
| 7 | 12.0 | Nonreactive |
| 8 | 339.9 | Reactive |
| 9 | 360.4 | Reactive |
| 10 | 12.8 | Nonreactive |
| 11 | 954.5 | Reactive |
| 12 | 962.4 | Reactive |

*Reactions correspond to those in Table 1.

### Example 2

### Amplification with NS3 Primer Set

A. NS3 Primer Set. The target sequence was amplified using the NS3 primer set (NS3 S1 and NS3 A1, SEQUENCE ID NOS 7 and 6, respectively) and haptenated with adamantane at their 5′ end as described in Example 1. The amplified product was detected using different hybridization probes as shown in TABLE 3 and haptenated with carbazole at their 3′ end using standard cyanoethyl coupling chemistry. Nonspecific amplification/hybridization was assessed using human placental DNA (hpDNA; Sigma, St. Louis, Mo.). Reactivity was assessed using human phpDNA or ribosomal RNA (rRNA; Boehringer Mannheim, Indianapolis, Ind.).

TABLE 4

| Re- | | | |
|---|---|---|---|
| action | Target | Primer | Detection Probe |
| 1 | 250 ng | NS3 A1 + NS3 S1 | NS3 CJ1 + NS3 CJ2 |
| | hpDNA | (SEQ ID NO 7 + SEQ | (SEQ ID NO 8 + |
| | | ID NO 6) | SEQ ID NO 9) |
| 2 | 10 fg | NS3 A1 + NS3 S1 | NS3 CJ1 + NS3 CJ2 |
| | NS3 plasmid | (SEQ ID NO 7 + SEQ | (SEQ ID NO 8 + |
| | | ID NO 6) | SEQ ID NO 9) |
| 3 | 1 pg | NS3 A1 + NS3 S1 | NS3 CJ1 + NS3 CJ2 |
| | NS3 plasmid | (SEQ ID NO 7 + SEQ | (SEQ ID NO 8 + |
| | | ID NO 6) | SEQ ID NO 9) |
| 4 | 30 pg | NS3 A1 + NS3 S1 | NS3 CJ1 + NS3 CJ2 |
| | NS3 plasmid | (SEQ ID NO 7 + SEQ | (SEQ ID NO 8 + |
| | | ID NO 6) | SEQ ID NO 9) |
| 5 | 500 ng | NS3 A1 + NS3 S1 | NS3 RM5 + NS3 RM6 |
| | rRNA | (SEQ ID NO 7 + SEQ | (SEQ ID NO 14 + |
| | | ID NO 6) | SEQ ID NO 15) |
| 6 | 106 | NS3 A1 + NS3 S1 | NS3 RM5 + NS3 RM6 |
| | NS3 RNA | (SEQ ID NO 7 + SEQ | (SEQ ID NO 14 + |
| | | ID NO 6) | SEQ ID NO 15) |
| 7 | 500 ng | NS3 A1 + NS3 S1 | NS3 RM1 + NS3 RM4 |
| | rRNA | (SEQ ID NO 7 + SEQ | (SEQ ID NO 10 + |
| | | ID NO 6) | SEQ ID NO 13) |
| 8 | 10 fg | NS3 A1 + NS3 S1 | NS3 RM1 + NS3 RM4 |
| | NS3 plasmid | (SEQ ID NO 7 + SEQ | (SEQ ID NO 10 + |
| | | ID NO 6) | SEQ ID NO 13) |
| 9 | 100 fg | NS3 A1 + NS3 S1 | NS3 RM1 + NS3 RM4 |
| | NS3 plasmid | (SEQ ID NO 7 + SEQ | (SEQ ID NO 10 + |
| | | ID NO 6) | SEQ ID NO 13) |
| 10 | 500 ng | NS3 A1 + NS3 S1 | NS3 RM2 + NS3 RM4 |
| | rRNA | (SEQ ID NO 7 + SEQ | (SEQ ID NO 11 + |
| | | ID NO 6) | SEQ ID NO 13) |
| 11 | 10 fg | NS3 A1 + NS3 S1 | NS3 RM2 + NS3 RM4 |
| | NS3 plasmid | (SEQ ID NO 7 + SEQ | (SEQ ID NO 11 + |
| | | ID NO 6) | SEQ ID NO 13) |
| 12 | 100 fg | NS3 A1 + NS3 S1 | NS3 RM2 + NS3 RM4 |
| | NS3 plasmid | (SEQ ID NO 7 + SEQ | (SEQ ID NO 11 + |
| | | ID NO 6) | SEQ ID NO 13) |
| 13 | 500 ng | NS3 A1 + NS3 S1 | NS3 RM3 + NS3 RM4 |
| | rRNA | (SEQ ID NO 7 + SEQ | (SEQ ID NO 11 + |
| | | ID NO 6) | SEQ ID NO 13) |
| 14 | 10 fg | NS3 A1 + NS3 S1 | NS3 RM3 + NS3 RM4 |
| | NS3 pasmid | (SEQ ID NO 7 + SEQ | (SEQ ID NO 11 + |
| | | ID NO 6) | SEQ ID NO 13) |
| 15 | 100 fg | NS3 A1 + NS3 S1 | NS3 RM3 + NS3 RM4 |
| | NS3 plasmid | (SEQ ID NO 7 + SEQ | (SEQ ID NO 11 + |
| | | ID NO 6) | SEQ ID NO 13) |

B. Description of the NS3 plasmid. PCR extension was performed using 5×EZ buffer (Perkin Elmer, Foster City, Calif.) which consisted of 250 mM Bicine, 575 mM postasium acetate, 40%(w/v) glycerol, pH 8.2, and Mn(oAc)2 at 2.5 mM final concentration. The nucleotides were at a concentration of 200 μM. The reaction conditions for TABLE 5 are shown in TABLE 5 below.

The NS3 plasmid (Clone pHGBV-C clone #1) was deposited at the American Type Culture Collection, 12301 Parklawn Drive, Rockville, Md. 20852 as of Nov. 8, 1994, under the terms of the Budapest Treaty and will be maintained for a period of thirty (30) years from the date of deposit, or for five (5) years after the last request for the deposit, or for the enforceable period of the U.S. patent, whichever is longer. The deposits and any other deposited material described herein are provided for convenience only, and are not required to practice the present invention in view of the teachings provided herein. pHGBV-C clone #1 was accorded A.T.C.C. Deposit No. 69711. The HGBV cDNA sequences in all of the deposited materials are incorporated herein by reference.

TABLE 5

| | Primer | Probe | Enzyme |
|---|---|---|---|
| Reactions | Concentration | Concentration | Concentration |
| 1-4* | 0.25 μM | 0.01 μM | 5 U rTth |
| 5,6** | 0.25 μM | .01 μM | 5 U rTth |
| 7-15*** | 0.25 μM | .005 μM | 5 U rTth |

*Cycling/hybridization conditions: 95° C. 2′ 1 cycle; 94° C. 1′/55° C. 1′/72° C. 1′ 30 cycles; 95° C. 5′, 15° C. soak;
**Cycling/hybridization conditions: 55° C. 30′, 94° C. 2′ 1 cycle; 94° C. 1′, 55° C. 1′, 72° C. 1′ 35 cycles; 97° C. 5′, 15° soak;
***Cycling/hybridization Conditions: 94° C., 2′ 1 cycle; 94° C. 1′, 55° C. 1′, 72° C. 1′ 35 cycles; 97°, 5′, 15°soak.
After maintaining the reaction mixture at 95° C. for 5 minutes, probe hybridization was accomplished according to the present invention by lowering the temperature of the amplification reaction 95° to 15° in 11 minutes according to regimen provided in Example 1:

Following amplification, reaction products were hybridized and detected on the Abbott LCx® system. These data are presented in TABLE 6. The data in TABLE 4 demonstrated specific amplification and detection of the HGBV target sequence.

TABLE 6

| Reaction* | LCx ® (c/s/s) |
|---|---|
| 1 | 33.2 |
| 2 | 1269.9 |
| 3 | 1888.3 |
| 4 | 1944.3 |
| 5 | 15.5 |
| 6 | 245.4 |
| 7 | 11.6 |
| 8 | 68.0 |
| 9 | 155.7 |
| 10 | 13.3 |
| 11 | 141.5 |
| 12 | 203.6 |
| 13 | 15.8 |
| 14 | 80.6 |
| 15 | 133.4 |

*Reactions correspond to those in Table 4.

### Example 3

### GB Serum Sample PCR/LCx® Parameters

“IVDU 300” was a sample known to contain the GB agent. It was tested as described hereinbelow. The negative control was normal serum.

A. HGBV 5′ NTR Detection.

The target sequences (TABLE 7) were PCR amplified using the primers (SEQ ID No. 1 and 2) and detection probes (SEQ ID No. 3 and 4) as described in Example 1. For this study, the primers were at a concentration of 0.25 mM (3.0×10¹³ molecules) and the detection probes were at a concentration of 0.01 mM (1.2×10¹² molecules.). In addition there was 0.025 units/ml (5 units total) of rTth DNA polymerase and 20 ng total of rRNA.

The reverse transcriptase reaction was performed for 60° C. for 30 minutes. The product was PCR amplified under the following cycling conditions: 94° C. for 1 min./55° C. for 1 min./72° C. for 1 min for 40 cycles. Next, the oligomer hybridization step was a 95° C., 5′, 15° C. soak. After maintaining the reaction mixture at 95° C. for 5 minutes, probe hybridization was accomplished according to the present invention by lowering the temperature of the amplification reaction 95° to 15° in 11 minutes according to the regimen provided in Example 1.

Following amplification, reaction products were detected on the Abbott LCx® system (available from Abbott Laboratories, Abbott Park, Ill.). The data from these experiments are presented in TABLE 7. The data in TABLE 7 demonstrated specific amplification and detection of the HGBV target sequence.

TABLE 7

| | | serum | LCx ® |
|---|---|---|---|
| sample | prep method | equivalents, ml | avg. c/s/s |
| control | QIAgen* | 0.25 | 24 |
| control | RNAzol B** | 2.5 | 26 |
| IVDU 300 | QIAgen | 0.25 | 251 |
| IVDU 300 | RNAzol B | 2.5 | 432 |

*QIAgen nucleic acid purification method obtained from QIAgen, Inc. (CA)
**RNAzol B nucleic acid purification method from Biotecx (Houston, TX)

B. HGBV NS3 Detection

The target sequences (TABLE 8) were PCR amplified using the primers (SEQ ID No. 6 and 7) and detection probes (SEQ ID No. 14 and 15) as described in Example 2. For this study, the primers were at a concentration of 0.25 mM (3.0×10¹³ molecules) and the detection probes were at a concentration of 0.01 mM (1.2×10¹² molecules.). In addition there was 0.025 units/ml (5 units total) of rTth DNA polymerase and 500 ng total of rRNA.

The reverse transcriptase reaction was performed at 64° C. for 10 minutes/62° C. for 10 minutes/60° C. for 10 min./58° C. for 10 min./56° C. for 10 min./54° C. for 10 min./52° C. for 10 min./50° C. for 10 min. The product was PCR amplified under the following cycling conditions: 94° C. for 1 min./55° C. for 1.5 min. for 40 cycles. Next, the oligomer hybridization step was a 95° C., 5′, 15° C. soak. After maintaining the reaction mixture at 95° C. for 5 minutes, probe hybridization was accomplished according to the present invention by lowering the temperature of the amplification reaction 95° to 15° in 11 minutes according to the regimen provided in Example 1.

Following amplification, reaction products were detected on the Abbott LCX® system (available from Abbott Laboratories, Abbott Park, Ill.). The data from these experiments are presented in TABLE 8. The data in TABLE 8 demonstrated specific amplification and detection of the HGBV target sequence.

TABLE 8

| | | serum | LCx ® |
|---|---|---|---|
| Sample | Prep method | equivalents, ml | avg. c/s/s |
| control | QIAgen* | 0.25 | 38 |
| control | RNAzol B** | 2.5 | 31 |
| IVDU 300 | QIAgen | 0.25 | 148 |
| IVDU 300 | RNAzol B | 2.5 | 373 |

*QIAgen nucleic acid purification method obtained from QIAgen, Inc. (CA)
**RNAzol B nucleic acid purification method from Biotecx (Houston, TX)

What is claimed is:

1. A method for detecting a target nucleic acid sequence comprising the steps of:
(a) forming a reaction mixture comprising nucleic acid amplification reagents, a hybridization probe, and a test sample suspected of containing nucleic acid comprising said target nucleic acid sequence, wherein
(i) said amplification reagents comprise at least one amplification primer comprising a nucleic acid sequence complementary to and capable of hybridizing with said sample nucleic acid such that the primer, upon extension by said amplification reagents, results in a nucleic acid comprising a nucleic acid sequence complementary to said target sequence, or complementary to a portion thereof;
(ii) said hybridization probe comprising a nucleic acid sequence which (a) is complementary to a portion of said sequence complementary to said target sequence and (b) is different from said primer nucleic acid sequence;
(b) subjecting said mixture to amplification conditions to generate at least one said nucleic acid comprising a nucleic acid sequence complementary to said target sequence;
(c) hybridizing said probe to said nucleic acid comprising a nucleic acid sequence complementary to said target sequence, so as to form a complex comprising said probe and said nucleic acid;
(d) detecting the presence of said complex as an indication of the presence of said target sequence in said sample.

2. The method of claim 1 wherein said probe and said primer have distinct temperatures such that the melt temperature of the probe is different from the melt temperature of the primer.

3. The method of claim 1 wherein the melt temperature of the primer is higher than the melt temperature of the probe.

4. The method of claim 1 wherein said primer comprises a detection label and said probe carries at least one capture label.

5. The method of claim 1 wherein said probe carries a capture label at said probes 3′ end and 5′ end.

6. The method of claim 1 wherein amplifying said mixture is accomplished with PCR.

7. A kit for detecting the presence of a nucleic acid sequence in a test sample comprising one or more suitable containers containing:
(a) a non-extendible hybridization probe which is complementary to said target sequence's complementary sequence; and
(b) an amplification primer which is complementary to said target sequence.


SEQUENCE LISTING (15 sequences)

SEQ ID NO: 1 - DNA (synthetic DNA), 21 bp; single-stranded, linear
  5′ Adamantane 1
cactgggtgcaagccccagaa

SEQ ID NO: 2 - DNA (synthetic DNA), 21 bp; single-stranded, linear
  5′ Adamantane 1
cactggtccttgtcaactcgc

SEQ ID NO: 3 - DNA (synthetic DNA), 15 bp; single-stranded, linear
  3′ Cabazole 15
agggttggtaggtcg

SEQ ID NO: 4 - DNA (synthetic DNA), 17 bp; single-stranded, linear
  3′ Carbazole 17
cacggtccacaggtgtt

SEQ ID NO: 5 - DNA (synthetic DNA), 18 bp; single-stranded, linear
  3′ Carbazole 18
cgcaacgacgcccatgta

SEQ ID NO: 6 - DNA (synthetic DNA), 26 bp; single-stranded, linear
  5′ Adamantane 1
ggnrmkrtyccyttttatgggcatgg

SEQ ID NO: 7 - DNA (synthetic DNA), 26 bp; single-stranded, linear
  5′ Adamantane 1
acnacnaggtcnccrtcyttgatgat

SEQ ID NO: 8 - DNA (synthetic DNA), 14 bp; single-stranded, linear
  3′ Carbazole 14
aggggggtcaaygc

SEQ ID NO: 9 - DNA (synthetic DNA), 14 bp; single-stranded, linear
  3′ Carbazole 14
gcytattayvgggg

SEQ ID NO: 10 - DNA (synthetic DNA), 15 bp; single-stranded, linear
  3′ Carbazole 15
caytcnaaggcggag

SEQ ID NO: 11 - DNA (synthetic DNA), 18 bp; single-stranded, linear
  3′ Carbazole 18
ygycaytcnaaggcggag

SEQ ID NO: 12 - DNA (synthetic DNA), 21 bp; single-stranded, linear
  3′ Carbazole 21
ttcygycaytcnaaggcggag

SEQ ID NO: 13 - DNA (synthetic DNA), 18 bp; single-stranded, linear
  3′ Carbazole 18
cattcctctggagcggat

SEQ ID NO: 14 - DNA (synthetic DNA), 16 bp; single-stranded, linear
  3′ Carbazole 16
ggggggtnaaygcyat

SEQ ID NO: 15 - DNA (synthetic DNA), 12 bp; single-stranded, linear
  3′ Carbazole 12
gcctattayagg